(19) United States
(12) Patent Application Publication
(10) Pub. No.: US 2015/0342618 A1
(43) Pub. Date: Dec. 3, 2015

(54) ULTRASOUND BONE CUTTING SURGICAL PROBE WITH DYNAMIC TISSUE CHARACTERIZATION

(71) Applicant: Michael N. Nguyen, Fremont, CA (US)

(72) Inventor: Michael N. Nguyen, Fremont, CA (US)

(21) Appl. No.: 14/723,300

(22) Filed: May 27, 2015

Related U.S. Application Data
(60) Provisional application No. 62/006,128, filed on May 31, 2014.

(51) Int. Cl. A61B 17/16; A61C 1/07; A61C 1/00; A61B 17/32; A61C 8/00

(52) U.S. Cl. CPC A61B 17/1626; A61B 17/1628; A61B 17/320068; A61B 17/1695; A61C 8/0092; A61C 1/0015; A61C 1/07; A61B 2017/00106

(57) ABSTRACT

An ultrasound bone cutting instrument with dynamic tissue characterization comprises a central control unit configured for generating low frequency and high frequency output signals and for receiving return signals, a hand-held probe containing an array of transducers and a preprocessing circuit, the transducers configured for converting the output signals into low frequency and high frequency ultrasound energy and for converting a portion of the ultrasound energy reflected from tissue areas in a target area into the return signals, and a cutting tip for cutting bone in the target area, wherein the central control unit is configured for determining characteristics of the tissues being approached by the cutting tip in response to the return signals.

### CROSS-REFERENCE TO RELATED APPLICATIONS

This application claims the benefit of U.S. Provisional Application No. 62/006,128, filed May 31, 2014.

### BACKGROUND

1. Field of the Invention

The invention is directed to orthopedic surgical instruments and more particularly to an improved ultrasound bone cutting and shaping surgical probe that provides dynamical feedback regarding characteristics of tissue being approached or operated on by the instrument.

2. Discussion of the Prior Art

In many types of osseous surgery, the surgeon usually has to deal with cutting an area of bone while at the same time not causing damage to adjacent soft tissues such as nerve trunks, blood vessels, delicate membranes, as well as malignant lesions. For example, in dental implant surgery the delicate membranes, nerve trunks and blood vessels must be avoided during bone cutting in order to assure the successful outcome of the procedure. While dental implant surgery will be used to demonstrate many technical aspects of the invention in this disclosure, it should be understood that scope of the invention is not restricted to dental implant surgery.

The sinus consists of a bone wall that is covered by a thin membrane known as the Schneiderian membrane. The underlying bone wall varies in thickness up to 20 mm thick depending on which side of the sinus the wall is located. When a tooth is extracted, the sinus floor bone can be as thin as 1-3 mm and in some cases may have eroded away completely.

In cases when the sinus floor bone is not thick enough to securely anchor a dental implant, bone thickness can be increased by lifting the SM so that external particulate bone, or a suitable substitute material, of a sufficient height and volume can be grafted onto the sinus floor.

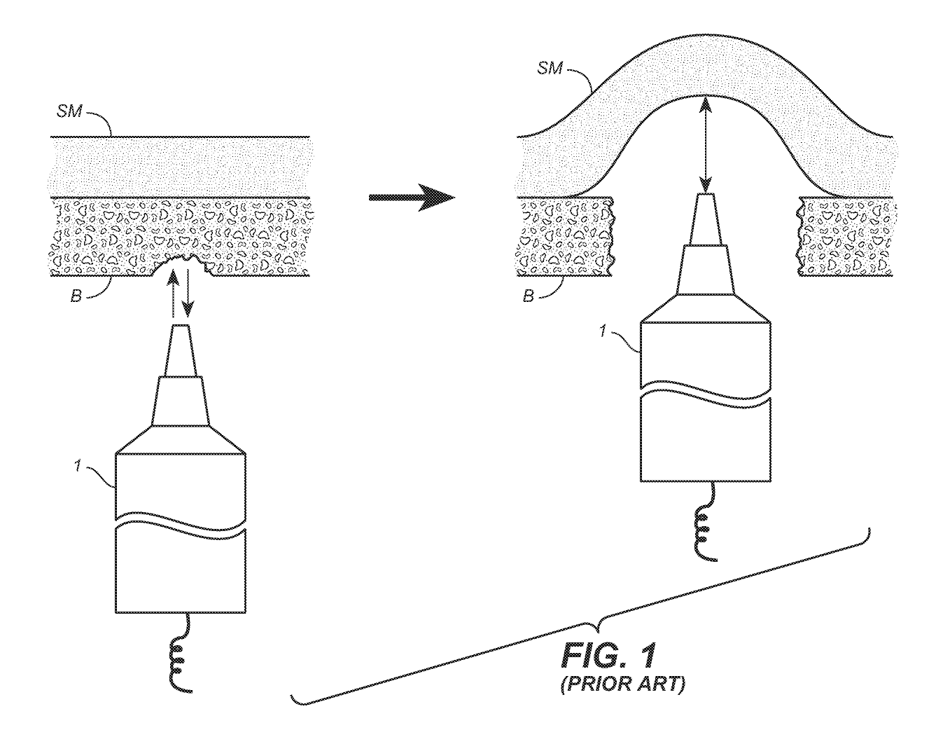

As shown in FIG. 1, the dental surgeon usually accesses the SM by cutting a window through the sidewall of the sinus or drilling a hole through the sinus floor bone B, taking care not to damage or perforate the SM. Under the existing state of the art, the sinus bone is typically cut or carved out using an ultrasound probe 1 that is not sensitive to the fragility of the delicate SM, frequently resulting in undesirable perforation of the SM.

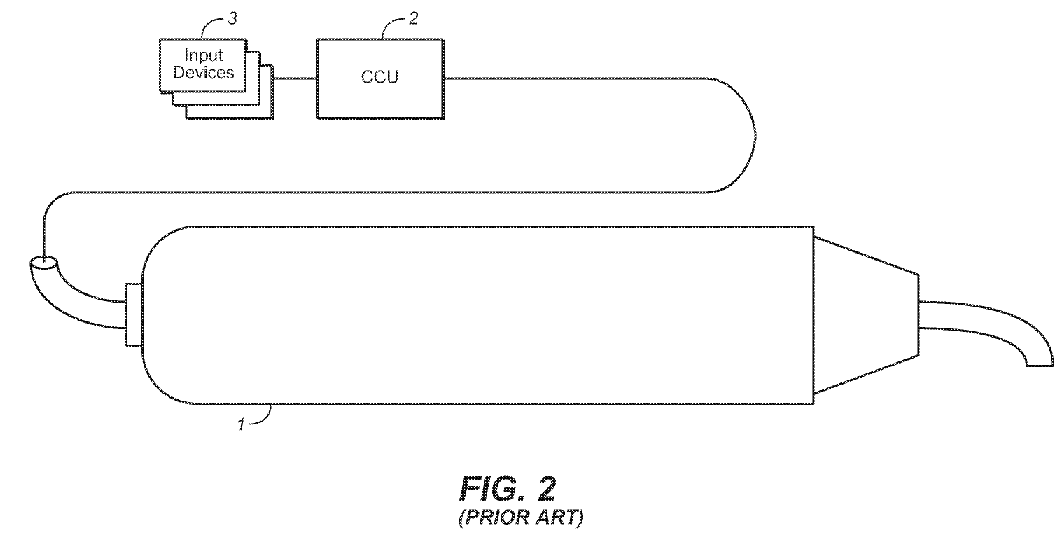

The prior art bone cutting instrument comprises a hand-held ultrasound probe 1 that is manipulated by the surgeon, a central control unit (CCU) 2, and user interface systems 3, as seen in FIG. 2. The probe consists of an internal set of transducers connected to a waveguide that transmits ultrasound energy to the probe end, commonly referred to as the “effector.” The CCU controls frequency of vibration and power by sending modulated signals through a driver circuit that excite the transducers to vibrate in a relatively low frequency range from approximately 20 kHz to 50 kHz. Vibration of the transducers causes the waveguide to vibrate which in turn causes the effector to vibrate. When the waveguide and probe length are properly coordinated with the wave length associated with the vibration frequency, a standing wave sets up in the waveguide that forms nodes and anti-nodes arranged along the waveguide so that the maximum vibrating energy is concentrated at the effector which can then be used to cut or emulsify bone with which it comes into contact, all the while avoiding damage to soft tissue. The probe also applies a constant stream of pressurized water to cool off bone that is being cut.

Instrument panels shown on a display allow the surgeon to select parameters that affect the ultrasound probe such as vibration frequency, power output, and water pressure. These parameters are typically keyed or programmed into the CCU via an input device such as a foot switch or keyboard before activating the ultrasound probe and they remain constant throughout operation of the ultrasound probe. If it becomes necessary to change parameters, the probe must first be deactivated before new parameters can be programmed or keyed in. When instrument parameters have been set, surgeon then directs the probe at the target to cut bone.

The prior art ultrasound probe is primarily focused on the ability of the unit to cut, scrape, or emulsify the target bone and relies heavily on the skill of the surgeon to advance deep into the targeted bone. Without information regarding the proximity and nature of the tissues being approached, the likelihood of damaging the tissues during the operation is increased. A notable attribute of the prior art probe is that information flow is unidirectional from the CCU to the probe in that controlling electrical signals sent from the CCU to the transducers only direct operation of the probe, whereas no information is sent back from the probe to the CCU. The prior art probe thus is a crude cutting instrument that provides little useful information to the surgeon.

### SUMMARY OF THE INVENTION

In the proposed design, an improved ultrasonic probe will be able to tell the operator the bone height or thickness remaining to be cut, the proximity of the cutting edge of the probe end to the SM, the thickness of the SM, whether the SM has been perforated, and the height and volume of space under the SM as it is being lifted—all precisely and dynamically during the operating procedure. The intelligent probe is also able to dynamically control the water stream to facilitate lifting of the SM at the optimum hydraulic pressure and duration.

In addition to dental surgery, the probe has application in many other branches of orthopedic surgery, such as spinal bone surgery, where for example, analogous to the SM, the orthopedic surgeon might wish to gently push away or lift surrounding nerve fiber or blood vessels from a certain bony structure.

An improved ultrasonic probe according to the invention is capable of dynamically cutting and simultaneously evaluating hard and soft tissues to avoid unnecessary damage to both, measuring bone thickness, measuring soft tissue thickness, controlling the cutting tip to optimize the desired outcome, controlling hydraulic pressure to achieve a desired pushing or lifting result, and operating in several modes tailored to the needs of the individual surgeon.

### BRIEF DESCRIPTION OF THE ILLUSTRATIONS

FIG. 1 shows the mechanical function of the hand piece of a prior art ultrasound bone cutting surgical probe being used to cut bone and lift the Schneiderian membrane.

FIG. 2 is a schematic diagram of a prior art ultrasound bone cutting surgical probe in communication with a central control unit and input devices.

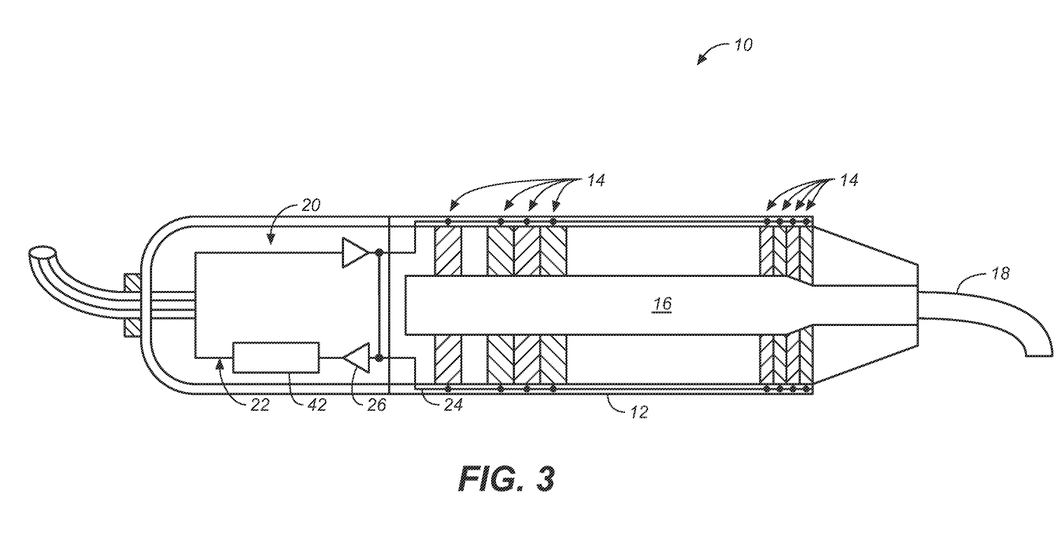

FIG. 3 is a schematic diagram of the hand-held probe of an improved ultrasound bone surgical instrument according to the invention.

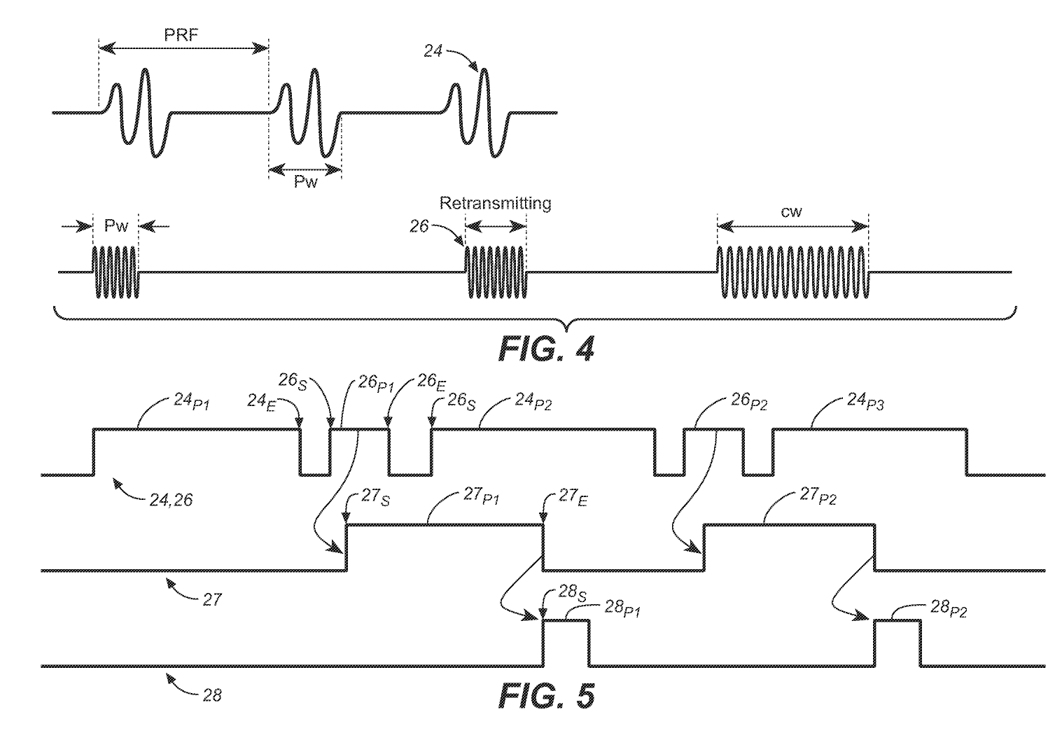

FIG. 4 is a diagram showing an exemplary pulse width and pulse repetition frequency of output signals transmitted from the central control unit.

FIG. 5 is a diagram showing operation of the probe in mixed mode.

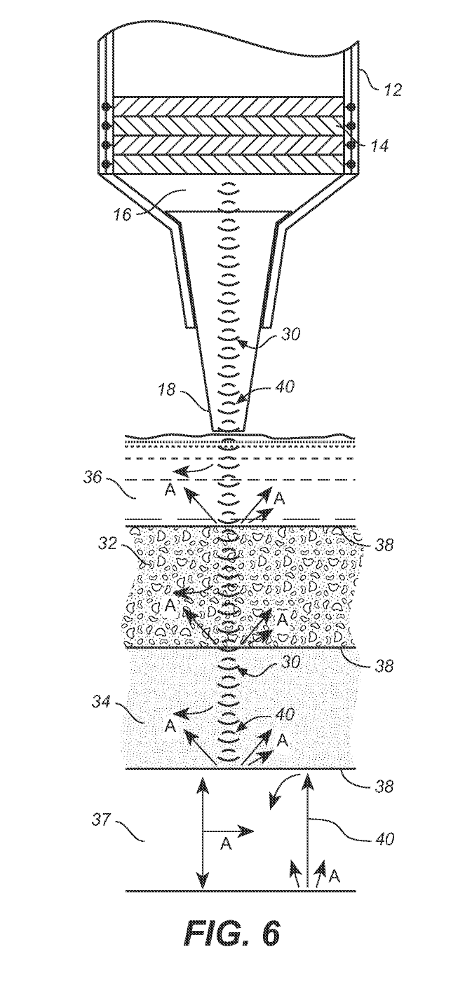

FIG. 6 is a diagram showing attenuation and reflection of a typical ultrasound wave through multiple layers of tissue and media.

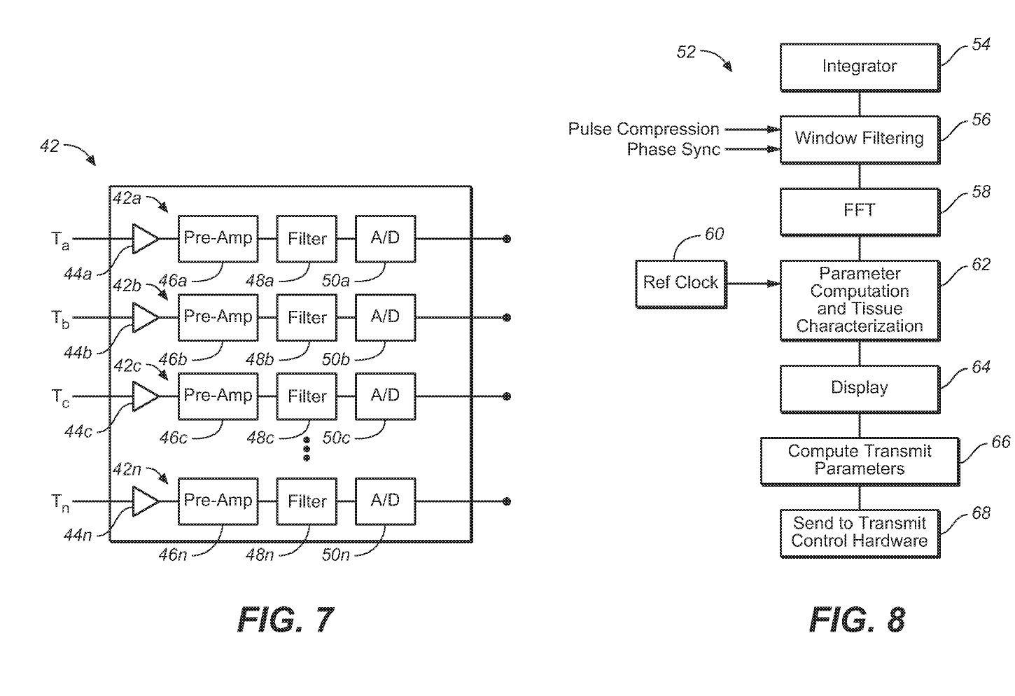

FIG. 7 is a flow chart of the steps for preprocessing the digital signal derived from the return pulse.

FIG. 8 is a flow chart of he steps for post-processing the digital signal derived from the return pulse.

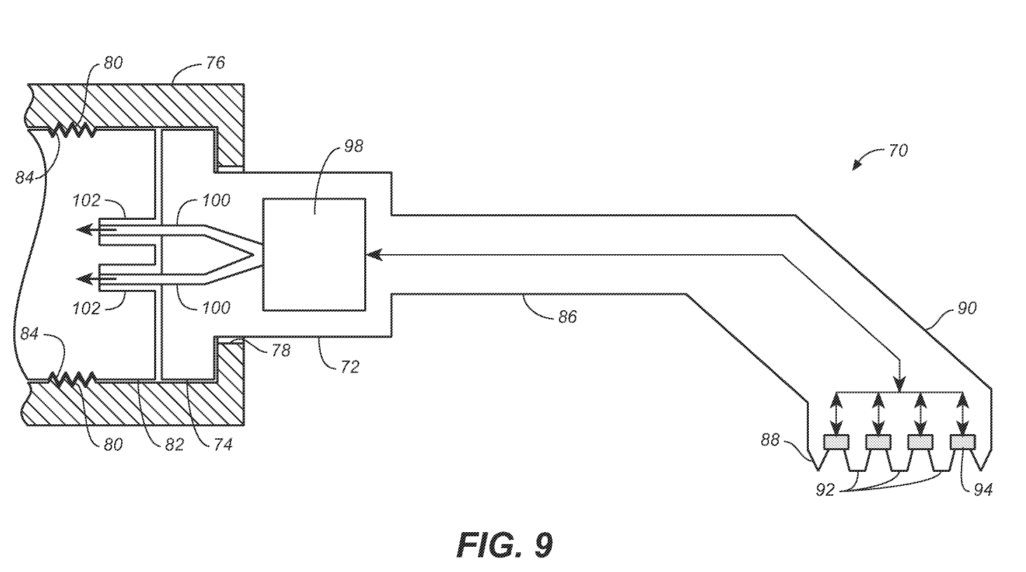

FIG. 9 is a schematic diagram showing an alternate embodiment of a cutting tip of a bone cutting surgical probe according to the invention.

### DETAILED DESCRIPTION OF THE ILLUSTRATED EMBODIMENT

An ultrasound bone cutting surgical probe according to the invention, referred to generally at numeral 10 in FIG. 3, includes a separate electronic circuit that dynamically senses and characterizes soft tissue being approached by the instrument while the probe is engaged in the bone cutting process. In particular, a high frequency pulse, operating in the range from 1 MHz to 20 MHz, is introduced into the probe in addition to the low frequency pulse used in the prior art.

The improved probe 10 comprises a housing 12 containing a plurality of transducers 14, a waveguide 16, a cutting tip 18, a driver circuit 20 and a sensing circuit 22. The housing 12 is designed to be gripped by hand and manipulated by the surgeon. The driver circuit 20 functions as described above with respect to prior art ultrasound probes, directing control signals from a CCU to excite the transducers 14 to vibrate in a low frequency range. The waveguide 16 transmits the vibrations from the transducers 14 to the cutting tip 18 which can then be used to cut, scrape or emulsify a targeted area of bone.

In the improved bone cutting surgical probe, the sensing signal 22, operating in a modulated higher frequency ultrasonic range between approximately 1 MHz to 20 MHZ is sent through the driver circuit 20 to the transducers 14 causing then to emit modulated higher frequency ultrasound energy.

FIG. 4 shows a simplified representation of a lower frequency control signal 24 and of a higher frequency sensing signal 26. The pulse width PW and the pulse repetition frequency PRF of low frequency signal 24 can be varied depending on power requirements for bone cutting. Increasing the PW and/or decreasing the PRF results in an increase in cutting power, and decreasing the PW and/or increasing the PRF results in a decrease in cutting power, Similarly, increasing the PW will increase the power of the sensing signal 26. Also, increasing the PRF of the sensing signal 26 results in an increase in the amount of time allowed to process and characterize the data received in response to the sensing signal 26. Each of these control signals can operate independently in a different time frame.

FIG. 5 shows a representative timeline indicating how transmission of a lower frequency signal control signal 24 can be time multiplexed with transmission of a higher frequency sensing signal 26, and a third signal 28, on the driver circuit 20 during a mixed frequency operation. On the first line, signal 24 powers bone cutting for a predetermined time until it is shut down at 24_{E}, immediately followed by starting of the sensing signal 26 at 26_{S}. On the second line, signal 27 indicates the detection at 27S of the received signal and therefore the start of processing of the received data. While the instrument is processing data indicating by the pulse 27, the sensing signal 26 can be shut down at 26_{E} to allow the next bone cutting control signal 24 to start at 24_{S}, as shown in the first line. As soon as the received data is analyzed and processed by the digital hardware and software algorithm, the control signal 27 is turned off at 27_{E}, and the data display signal 28 is turned on at 28_{S} as shown in the third line of FIG. 5. The whole cycle is repeated again until the satisfactory result is achieved.

With reference now to FIG. 6, it can be seen that ultrasound energy 30 at both higher and lower frequencies is transmitted in modulated pulses along the waveguide 16 to the cutting tip 18 from which it continues to forward-adjacent tissue layers such as bone 32, soft tissues 34, liquid 36, other connective tissue types, or air 37. Ultrasound energy is reduced due to the mechanisms of absorption, reflection, scattering and diffraction at different material layers and boundaries, as indicated by arrows A. However, at each boundary 38 between different tissue type layers, e.g., bone-to-soft tissue, soft tissue-to-air or water-to-soft tissue, a small portion 40 of the ultrasound energy is reflected back through intervening tissues to the cutting tip 18, and the waveguide 16. The reflected ultrasound energy 40 is converted by the transducers 14 into an electrical signal for transmission back to the CCU through sensing circuit 22. The CCU receives the signal, conditions, analyzes, and parameterizes it and then dynamically shows information derived from the signal on the display. The reflected pulses carry critical information specific to the tissue being encountered, including tissue type and physical characteristics such as its thickness, density and elasticity. After the underlining tissues, e.g., hard bone, soft membrane or nerve fibers, are analyzed, the CCU uses this information to control the transmitting electronics to affect the probe behavior such as by varying the frequency of vibration of the probe. It also controls the pressure of the jet of water being applied to the tissues by sending controlling signals to the hydraulic pump, and is capable of taking an image of the tissues being approached by the probe end.

The high frequency pulse thus enables the probe to accurately sense boundaries between different tissue types, characterize the tissue type, and compute the tissues' thickness and spatial relationship with adjacent tissues. This enables the surgeon to react to dynamic conditions during the procedure and control subsequent transmitting protocols to the probe. When performing a sinus lift procedure in dentistry, this information gives the surgeon greater control over the procedure and enables a more precise approach to and lifting of the SM.

Mode of Operation

In one aspect of the invention, the ultrasonic probe is capable of operating in single frequency or mixed modes.

Single Frequency Mode

In single frequency mode, the probe operates in either the low frequency range or the high frequency range. When operating at lower frequencies from 20 kHz to 50 kHz, the is devoted to bone cutting and reflected lower frequency ultrasound energy can be used to detect the depth of bone being approached or cut and thereby provide some warning when approaching the SM. Nevertheless, the surgeon is operating without useful information regarding the character and types of tissues being approached.

When operating at higher frequencies from 1 MHz to 50 MHz, the reflected ultrasound energy is used to measure information that can indicate tissue type and physical characteristics such as thickness, density, rigidity, tissue type, and bone-to-tissue separation height.

Mixed Mode

In mixed mode, the probe operates simultaneously in both lower and higher frequencies in a time multiplexing protocol as discussed above. Mixed mode thus combines the ability to detect the depth of bone being operated on by the probe with the ability to measure and evaluate the fluid and connective tissue media being approached, including the integrity of the SM or adjacent nerve fibers, the height and volume of an SM being lifted, and the ability to display an image of the lifted SM. When operating in mixed mode, the ultrasound probe acts both as a bone cutting tool and as a sensing tool that detects, characterizes and displays information regarding forward-adjacent hard and soft tissues.

Signal Detection

As mentioned above, the power of the ultrasound wave is attenuated significantly as it travels from the transmitting transducers 14, through the waveguide 16, toward and beyond the cutting tip 18 of the hand piece 12, and to the desired target, and as it is reflected back from the target to the hand piece, as shown in FIG. 6. Therefore, it is important to reduce the distance traveled by the ultrasound energy from the transducer to the target. As shown in FIGS. 3 and 6, the transmitting and receiving transducers 14 can be located inside the housing body of the hand piece, but as dose to the cutting tip as possible. Or, as shown in another embodiment of the invention in FIG. 9, discussed in greater detail below, the transmitting and receiving transducers 94 can be located at the cutting tip 70. By locating the transducer 94 at the cutting tip 70 instead of within the body housing, attenuation of the ultrasound energy is significantly reduced by an order of magnitude of approximately −40 dB round trip, assuming the distance between transducers 14 to the cutting tip is about 4 cm and the operating frequency is approximately 5 MHZ.

As the ultrasound energy travels beyond the cutting tip, it encounters water, bone, soft tissue (such as the Schneiderian membrane), and air layers before reflecting back to the sensing transducers. The bone layer is responsible for most of the power attenuation of the ultrasound waves. At 5 MHz, and assuming average bone thickness of 2 mm, the round trip power lost due to bone layer is about −40 dB. Therefore, when the transducers 14 are located inside the body housing, the total round trip attenuation is about −80 dB at 5 MHz—the sum of power lost inside the housing body and in the external environment (mostly due to bone mass). But when the transducers 94 are located at the cutting tip 70, the total round trip ultrasound attenuation is only about −40 dB—an order of magnitude improvement. Existing commercial chips or chip sets with power gain exceeding 90 dB are available to allow the successful design of the detection front-end hardware. As the sensing frequency increases due to resolution requirements, the ultrasound attenuation also increases. Therefore, a significant advantage of locating the sensing transducers at the probe's cutting tip is that the sensing frequency can be increased up to 50 MHz or higher. This will vastly increase the resolution of the tissue under investigation and, therefore, the resolution of the 2-dimensional gray scale images shown in the display, Such a high level of resolution is also needed in Doppler signal processing to investigate motion within tissues themselves such as blood flow within a blood vessel.

To maximize the ability to pick up the returning ultrasound energy, multiple transducers may be used and their combined energies summed to increase the overall detected signal level. These transducers are placed close together in a piggy back configuration and may be spaced apart distance from each other corresponding to the phase shifting of the returning ultrasound waves. The total detected energy is, therefore, the sum of all these phase-shifted signals at the output of the piezoelectric elements, There may be a plurality of piezoelectric elements depending on the transducer size and space available in the hand piece. However, it is anticipated that the number will usually be from 2 to 8, but possibly higher. The piezoelectric transducers can be the same as or integrated with the transducer that is transmitting the low frequency high power bone cutting energy, but locating them at or as close as possible to the cutting tip will minimize attenuation of the ultrasound energy.

The return ultrasound signal amplitude is preamplified in the preprocessing stage to significantly increase the signal-to-noise ratio and thereby increase the chance of detection. As will be familiar to those of skill in the art, such amplifiers are implemented by very low noise anti-alias filters. The ultrasound signal is then converted into a digital signal by an analog-to-digital (A/D) converter. The A/D converter is located immediately inside the hand piece housing, as shown in FIGS. 3 and 7, or in the preprocessing circuit 98 at the cutting tip, as shown in FIG. 9. By digitizing the ultrasound signal before sending it to the CCU via a long cable, the signal amplitude and strength are free from systemic noise or signal degradation.

To further improve detection capability, a digital filter is implemented in the CCU to significantly increase the detection dynamic range. A Doppler filter is used to detect very low signal energy within the very “noisy” environment. A digital filter bank may be implemented to detect and discriminate between the main lobe carrier frequency and other side lobe tissue-specific frequencies or noises.

Hardware and software design and implementation

Receiving Preprocessing Section

According to the illustrated embodiment of the invention, a preprocessing circuit 42 is located inside the hand piece housing 12, as seen in FIG. 2. As shown in FIG. 7, the preprocessing circuit 42 detects returning signals corresponding to ultrasound energy reflected from tissues in the target area received from each of a plurality of piezoelectric transducers Tₐ-Tₙ) on multiple channels 42ₐ-42ₙ. The returning signals are pre-processed and pre-conditioned in the pre-processing circuit 42 before being sent to the CCU for further analysis. Those of skill in the art will understand that the number of channels may vary from 4 to 8, 16, 32 or 64 depending on the specific application and the hardware integration technique being employed. Each channel is dedicated to one of the transducers T and consists of a receiving driver 44, analog pre-amplifier 46, an anti-alias analog filter 48 and an A/D converter 50. The preamplifier 46 is a high gain-low noise amplifier to increase signal detection. The anti-alias band pass filter 48 is used to reject undesirable frequency components outside the desirable detection range. Finally the A/D converter 50 digitizes the amplified signal. The ultrasound signals received on each channel have a phase relationship that allows them to be constructively added together in a signal integrator to increase the chance of signal detection.

One advantage of having the ND converter 50 located inside the hand piece housing 12 is that further loss of the ultrasound signal can be minimized as it travels through the long cable to the CCU, Suitable specialized multi-channel low noise receiver chips that operate in a dynamic range of approximately 90 dB are commercially available.

The preprocessed digital signals from each channel 42ₐ-42ₙ can be summed by a signal integrator 52 located either in the preprocessing circuit 42 or in the post-processing circuit located 54 within the CCU, depending on the availability of space within the hand piece housing 12 and the size of the interconnecting cable. See FIG. 8. At the signal integrator 52, the digitized signals from all channels are added together according to phase to increase the strength of the ultrasound signal.

Receiving Post-Processing

Referring now to FIG. 8, the post-processing circuit 52, located in the CCU and implemented by various hardware circuits and software algorithms, is used to enhance signal detection and characterization. As mentioned above, the integrator 54 may be located in the preprocessing circuit or included in the post-processing circuit 52. After the preprocessing digitized signal is integrated, it is then subjected to a digital band pass filter with window weighing at 56 to enhance signal-to-noise ratio. A Hilbert transformation filter can be used to derive a more useful analytic representation of he signal Pulse compression can also be used to increase power o enhance the returning signal.

A Fast Fourier Transform (FFT) is implemented at 58 to allow tissue characterization at 62. This is a process-intensive algorithm that can be implemented with hard-wire circuitry or high-speed chip sets. A reference dock is used at 60 to track signal timing in order to compute the spatial relationship, i.e., thickness, between different tissue types. One single pulse may initiate several detections each of which corresponds to several different medium layers. The derived data and a visual image are presented on a display at 64 to provide the surgeon with as much information as possible. Finally, the CCU computes transit parameters at 66 to control the power of the transducers dynamically at 68.

To identify the type of medium, e.g., bone, liquid, soft tissue or air space, the detected return signal is subjected to further digital signal processing 62. A digital filter bank with 8 points, 16 points, 32 points, 64 points, 128 points or 256 points may be needed to characterize the medium type. Since these are very process-intensive operations, the hardware implementation must be fast enough to work, but do so without interrupting the time line or the bone-cutting mixed mode operation. The hardware implementation may include the use of general purpose digital signal processors or specialized FFT signal processing chips or chip sets. A continuous wave Doppler mode may be implemented to identify soft tissue motion as it is pushed or lifted by the hydraulic pressure. In this mode, the main lobe of the filter bank is the operating frequency. By analyzing the phase velocity between the filter banks, soft tissue spatial characteristics can be computed.

After all data is computed, the CCU sends a refresh-display content command to the screen at 64, reflecting the current status of the hard and soft tissues being operated on. The content displayed includes at least the remaining bone thickness, the distance to the critical soft tissue layer, the volume or height of the lifted soft tissue layer, and tissue characteristics such as tissue density, elasticity and the velocity of blood in a blood vessel. The displayed data provides valuable dynamic feedback which helps the surgeon make decisions affecting the operation because the surgical progress can directly and dynamically be observed.

Based on the computed sensing data, and together with the required input parameters from the operating surgeon, the CCU computes the necessary transmitting parameters at 66 such as amplitude, pulse width and pulse repetition frequency of the low frequency signals in order to adjust cutting power. These transmit parameters are sent to transmit control hardware at 68 to control the power of the transmitting transducers of the hand piece. If the surgeon pushes the cutting tip too hard into the critical soft tissue structure, the cutting probe will either reduce the output power or completely stop automatically and dynamically to avoid damaging to the critical tissues. A crucial advantage of the invention thus is that the information gained from the returning ultrasound energy is used to control the probe to prevent the surgeon from inadvertently causing damage to the underlining critical soft tissue, such as the SM, nerve trunks, or blood vessels.

With reference now to FIG. 9, another embodiment of the invention is described which comprises a modular ultrasound tip 70 having an integrated array of transducers. The tip 70 comprises an attachment base 72 having an outwardly extending retaining flange 74. A lock nut 76 fits over and around the attachment base 72, capturing the retaining flange 74 with an inwardly extending retaining Hp 78 as shown, Threads 80 on the exterior surface of the hand piece 82 match threads 84 on the interior of the lock nut 76, such that tightening the lock nut 76 secures the attachment base 72 to the hand piece 82.

A tip arm 86 extending forward from the attachment base 72 includes a cutting head 88 on its distal end 90. The cutting head 88 includes an array of cutting teeth 92 interspersed between which is disposed an array of transducers 94. The transducers 94 are arranged to have a predetermined phase relationship with each other to allow implementation of a multi-channel design to enhance signal detection. The transducers are in this manner disposed as close as possible to the tissues being approached by the cutting head 88.

In one aspect of the invention, the transducers 94 located in the cutting head 88 are higher frequency transducers dedicated to transmitting and receiving high frequency ultrasound energy. A significant advantage of locating the high frequency transducers at the end 90 of the tip 70 is that ultrasound energy reflected from the target tissues can be picked up with minimized attenuation which otherwise would occur from travel of the energy through components of the probe if the transducers where located in the probe body.

A pre-processing and pre-amplifying circuit 98 is housed in the attachment head to reduce noise and interference and improve signal integrity. The preamplifier 98 is electrically connected to rearward-facing pins 100 which can be plugged into sockets 102 provided in the hand piece 82. The sockets 102 are electrically connected to the CCU permitting signals to be transmitted between the CCU and the tip 70, thus allowing sensor signals to be sent from the tip to the CCU, and instructions to be sent from the CCU to the tip.

In mixed mode operation, discussed above, low frequency signals may be sent to transducers located in the probe body and high frequency signals may be sent to the transducers located in the cutting tip.

The immediate proximity of the transducers 94 in the cutting head 88 improves the quality of signals being reflected from the target tissues, and the ability to detach the tip 70 from the hand piece 82 permits modular attachment of a plurality of tips having different cutting heads to the hand piece. As mentioned above, this allows the sensing frequency to be increased up to 50 MHz and beyond thereby significantly increasing the sensitivity of the instrument with respect to tissue under investigation and enabling dynamic highly detailed tissue imaging.

The hardware and software design of the invention has distinct advantages over existing art ultrasonic surgical bone cutting probes. The new ultrasound probe provides a much more intelligent receiving path and a more robust transmitting mechanism with more desirable outcome not available using existing systems.

The new ultrasonic probe incorporates additional electronic circuitry and may also incorporate additional specialized piezoelectric transducers into the current design of the commercial devices.

There have thus been described and illustrated certain embodiments of a new ultrasound bone cutting surgical instrument according to the invention. Although the present invention has been described and illustrated in detail, it should be clearly understood that the disclosure is illustrative only and is not to be taken as limiting, the spirit and scope of the invention being limited only by the terms of the appended claims and theft legal equivalents.

1. An ultrasound bone cutting surgical instrument comprising:
a central control unit configured for generating one or more output signals and for receiving one or more return signals,
a power source for energizing said central control unit, and
a hand-held probe body in communication with said central control unit, said probe body comprising an array of transducers and a cutting tip,
said array of transducers configured for converting said one or more output signals into ultrasound energy, said cutting tip configured for vibrating at frequencies conducive to cutting to bone in a target area in response to said ultrasound energy, said target area comprising one or more tissue layers,
wherein, said cutting tip is further configured for receiving a portion of said ultrasound energy reflected from said one or more tissue areas and transmitting said reflected ultrasound energy to said array of transducers,
said array of transducers is further configured for converting said reflected ultrasound energy into said one or more return signals, and
said central control unit is further configured for determining one or more characteristics of said one or more tissue layers in response to said one or more return signals.

2. The ultrasound bone cutting surgical instrument of claim 1 wherein:
said probe body contains a wave guide coupled to said array of transducers, said cutting tip is detachably attached to said wave guide and extends from said probe body, said wave guide configured for transmitting said ultrasound energy from said array of transducers to said cutting tip and for transmitting said reflected ultrasound energy from said wave guide to said array of transducers.

3. The ultrasound bone cutting surgical instrument of claim 1 wherein:
said one or more output signals include a driver signal having a lower frequency between approximately 20 kHz and 50 kHz.

4. The ultrasound bone cutting surgical instrument of claim 3 wherein:
said central control unit is configured for detecting the depth of bone being approached by the cutting tip of said probe in response to said driver signal.

5. The ultrasound bone cutting surgical instrument of claim 1 wherein:
said one or more output signals include a sensor signal having a higher frequency between approximately 1 MHz and 20 MHz.

6. The ultrasound bone cutting surgical instrument of claim 5 wherein:
said central control unit is configured for detecting the type and physical characteristics of the one or more tissue layers in said target area in response to said sensor signal.

7. The ultrasound bone cutting surgical instrument of claim 1 wherein:
said one or more output signals comprise a multiplexed signal including a driver signal having a lower frequency between approximately 20 kHz and 50 kHz, and a sensor signal having a higher frequency between approximately 1 MHz and 20 MHz.

8. The ultrasound bone cutting surgical instrument of claim 1 wherein:
said central control unit is configured for detecting the depth of bone being approached by the cutting tip of said probe in response to said one or ore return signals.

9. The ultrasound bone cutting surgical instrument of claim 8 wherein:
said central control unit is configured for detecting the type and physical characteristics of the one or more tissue layers in said target area in response to said one or more return signals.

10. The ultrasound bone cutting surgical instrument of claim 9 wherein:
said one or more tissue layers include the Schneiderian Membrane of the maxillary sinus, and
said central control unit is configured for detecting the height, volume and integrity of the Schneiderian Membrane.

11. The ultrasound bone cutting surgical instrument of claim 1 wherein:
said probe includes a pressurized stream of water for directing at the tissues in the target area, and
said central control unit is further configured for adjusting the hydraulic pressure of said water stream in response to said one or more return signals.

12. The ultrasound bone cutting surgical instrument of claim 1 wherein:
the probe body contains a pre-processing circuit, said pre-processing circuit including an A/D converter for converting said one or more return signals from analog to digital.

13. The ultrasound bone cutting surgical instrument of claim 12 wherein:
said pre-processing circuit is configured for receiving, amplifying, and filtering said one or more return signals.

14. The ultrasound bone cutting surgical instrument of claim 13 wherein:
said array of transducers comprises a plurality of transducers spaced apart a distance corresponding to the phase shift of said reflected ultrasound energy waves, each of said plurality of transducers configured for converting a portion of said reflected ultrasound energy into a return signal, and
said pre-processing circuit includes a plurality of channels, each of said a plurality of channels configured for receiving, amplifying, and filtering the return signal received from one of said plurality of transducers.

15. The ultrasound bone cutting surgical instrument of claim 14 wherein:
said pre-processing circuit includes an integrator configured for summing the pre-processed return signals received from said plurality of channels.

16. The ultrasound bone cutting surgical instrument of claim 1 wherein:
said cutting tip includes an array of cutting teeth, and
said array of transducers is interspersed with said array of cutting teeth.

17. The ultrasound bone cutting surgical instrument of claim 1 wherein:
said cutting tip includes one or more pre-processing circuits, each of said one or more pre-processing circuits configured for receiving, amplifying, and filtering said one or more return signals and for converting said one or more return signals from analog to digital.

18. The ultrasound bone cutting surgical instrument of claim 1 wherein:
said array of transducers includes first and second arrays of transducers, said first array of transducers configured for generating low frequency ultrasound waves, said second array of transducers configured for generating high frequency ultrasound waves.
said probe comprises a hand-held housing containing a wave guide, said first array of transducers coupled to said wave guide, said cutting tip detachably attached to said wave guide and extending from said housing, said wave guide configured for transmitting said ultrasound energy from said first array of transducers to said cutting tip,
said wave guide further configured for transmitting said reflected ultrasound energy from said cutting tip to said first array of transducers,
said cutting tip including an array of cutting teeth, and said second array of transducers interspersed with said array of cutting teeth, said second array of transducers configured for converting said reflected ultrasound energy into said one or more return signals.

19. An ultrasound bone cutting surgical instrument comprising:
a central control unit configured for generating one or more output signals and for receiving one or more return signals,
a power source for energizing said central control unit, and
a probe body and a cutting tip, said housing containing an array of transducers and a wave guide coupled to said array of transducers, said array of transducers in communication with said central control unit, said cutting tip detachably attached to said wave guide and extending from said probe body,
said array of transducers configured for converting said one or more output signals into ultrasound energy,
said wave guide configured for transmitting said ultrasound energy from said array of transducers to said cutting tip,
said cutting tip configured for vibrating at frequencies conducive to cutting bone in a target area in response to said ultrasound energy, said target area comprising one or more tissue layers,
wherein, said cutting tip is further configured for receiving a portion of said ultrasound energy reflected from said one or more tissue areas and transmitting said reflected ultrasound energy to said wave guide,
said wave guide is further configured for transmitting said reflected ultrasound energy from said wave guide to said array of transducers,
said array of transducers is further configured for converting said reflected ultrasound energy into said one or more return signals, and
said central control unit is further configured for determining one or more characteristics of said one or more tissue layers in response to said one or more return signals.

20. An ultrasound bone cutting surgical instrument comprising:
a central control unit configured for generating one or more output signals and for receiving one or more return signals,
a power source for energizing said central control unit, and
a hand-held probe body containing a first array of transducers in communication with said central control unit,
a cutting tip including an array of cutting teeth, and a second array of transducers interspersed with said array of cutting teeth and in communication with said central control unit,
said first array of transducers configured for converting said one or more output signals into ultrasound energy,
said cutting tip configured for vibrating at frequencies conducive to cutting bone in a target area in response to said ultrasound energy, said target area comprising one or more tissue layers,
wherein, said second array of transducers is configured for converting said reflected ultrasound energy into said one or more return signals, and
said central control unit is further configured for determining one or more characteristics of said one or more tissue layers in response to said one or more return signals.
